(19) United States
(12) Patent Application Publication
(10) Pub. No.: US 2021/0100868 A1
(43) Pub. Date: Apr. 8, 2021

(54) COMPOSITIONS AND METHODS FOR TREATING OR PREVENTING GASTROINTESTINAL DAMAGE ASSOCIATED WITH NSAID THERAPY

(71) Applicant: NORTH CAROLINA STATE UNIVERSITY, Raleigh, NC (US)

(72) Inventor: Anthony BLIKSLAGER, Raleigh, NC (US)

(21) Appl. No.: 16/982,114

(22) PCT Filed: Mar. 19, 2019
(86) PCT No.: PCT/US2019/022886

§ 371 Date: Sep. 18, 2020

Related U.S. Application Data
(60) Provisional application No. 62/644,721, filed on Mar. 19, 2018.

(51) Int. Cl. A61K 38/08; A61P 1/04

(52) U.S. Cl. CPC A61K 38/08; A61P 1/04

(57) ABSTRACT

The present invention provides compositions and methods for treating or preventing gastrointestinal damage associated with acute or chronic non-steroidal anti-inflammatory drug (NSAID) therapy, including, for example, NSAID-induced gastropathy or enteropathy. In various embodiments, the invention relates to administering a regimen of larazotide (or a derivative) to a patient to repair or prevent injury to mucosal tissue of the gastrointestinal epithelium. In accordance with the invention, larazotide functions to repair injured mucosal tissue or prevent injury to mucosal tissue in a patient undergoing acute or chronic NSAID therapy.

### FIELD OF THE INVENTION

The present invention provides compositions and methods for treating or preventing gastrointestinal damage associated with NSAID therapy, such as NSAID-induced gastropathy or enteropathy.

### CROSS-REFERENCE TO RELATED APPLICATIONS

This application claims the benefit of U.S. Provisional Application No. 62/644,721, filed on Mar. 19, 2018, the entire contents of which are incorporated herein.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (Filename: “INN-015PC_ST25.txt”; Date created: Mar. 19, 2019; File size: 855 Bytes).

### BACKGROUND

Non-steroidal anti-inflammatory drugs (NSAIDs) are some of the most commonly prescribed drugs in the world to relieve pain, fever and inflammation. NSAIDs are prevalent in the management of both acute and chronic pain and inflammation, including for chronic pain associated with osteoarthritis, musculoskeletal conditions, and various autoimmune diseases.

However, when NSAIDs are administered as acute or chronic therapy, patients can develop gastrointestinal mucosal damage, including conditions known as NSAID-induced gastropathy or enteropathy. These conditions are characterized by damage to intestinal mucosal tissue and gastrointestinal complications, such as ulcerations, perforations, bleeding, formation of diaphragm-like strictures, and/or obstruction. NSAIDs undermine the integrity of the gastrointestinal epithelium by inhibiting the production of prostaglandins that have a cytoprotective role in the gastrointestinal mucosa. Resulting increased permeability of the GI epithelium allows toxins and microorganisms to breach the epithelial barrier and have unimpeded access to the systemic circulation.

Accordingly, there remains a need for effective therapies for treating and/or preventing gastrointestinal injury associated with acute or chronic NSAID use.

### SUMMARY OF THE INVENTION

The present invention provides compositions and methods for treating or preventing gastrointestinal damage associated with acute or chronic non-steroidal anti-inflammatory drug (NSAID) therapy, including, for example, NSAID-induced gastropathy or enteropathy. In various embodiments, the invention relates to administering a regimen of larazotide (or a derivative) to a patient to repair or prevent injury to mucosal tissue of the gastrointestinal epithelium. In accordance with the invention, larazotide functions to repair injured mucosal tissue or prevent injury to mucosal tissue in a patient undergoing acute or chronic NSAID therapy.

In some embodiments, the patient receives chronic NSAID therapy for chronic pain or for prevention of thrombosis. Administration of larazotide or derivative in these patients can reduce gastrointestinal damage associated with the chronic NSAID therapy. As shown herein, the mechanism of larazotide-induced recovery of intestinal mucosa is not dependent on prostaglandin production, and thus larazotide (or derivative) can be effective even in a condition of prostaglandin depletion in the gastrointestinal mucosa.

In some embodiments, the patient is exhibiting one or more symptoms of NSAID-induced gastropathy or enteropathy, or gastrointestinal mucosal tissue damage, including, but not limited to ulcer, bleeding, perforation, obstruction, epigastric pain, nausea, indigestion, constipation, and abdominal distension. In some embodiments, the patient exhibits one or more symptoms of side effects related to acute NSAID use, including, but not limited to, gas, bloating, heartburn, stomach pain, nausea, vomiting, and diarrhea.

In various embodiments, the larazotide is administered in a sustained release or controlled release formulation. The sustained release or controlled release formulation avoids accumulation of inactive fragments that may act as competitive inhibitors. For example, the formulation may comprise or deliver and/or functionally release from 0.5 to about 5 mg of larazotide or derivative. In various embodiments, the sustained release or controlled release formulation contains at least 1 mg or at least 2 mg of larazotide or derivative. The sustained or controlled release formulation may functionally release peptide over the course of at least about 2 hours or over the course of at least about 3 hours, as quantified using simulated intestinal fluid for example.

In various embodiments, larazotide is formulated as a plurality of particles that release larazotide at different times in intestinal fluid, or at different locations in the gastrointestinal tract. In other embodiments, the formulation releases larazotide in a form that provides for a local sustained release at one or more locations, including sustained release from particles, gels, emulsions, or biodegradable matrix. In some embodiments, the sustained or controlled release composition begins to release peptide starting within about 5 minutes to about 30 minutes of exposure to simulated intestinal fluid, with release of peptide continuing for at least about 180 minutes of exposure to simulated intestinal fluid. Release profiles can be prepared, for example, using particles with different enteric polymer coats and/or different thicknesses of the polymer coats.

In some embodiments, the formulation releases larazotide or derivative in simulated gastric fluid. Such formulations will release larazotide or derivative in the stomach upon administration. For example, a population of peptide-coated particles can be employed having an outer coating that disintegrates in simulated gastric fluid.

Various formulations can be employed to deliver the larazotide or derivative to a location of interest. For example, the compositions may be formulated for targeted delivery to the gastrointestinal tract including the stomach, small intestine, large intestine and rectum including all subsections thereof. By targeting release of larazotide or derivative in the affected region(s) (e.g. stomach, duodenum, jejunum, ileum, colon transversum, colon descendens, colon ascendens, colon sigmoidenum and cecum), tight junction integrity at various portions of the GI can be improved or protected during NSAID therapy.

In accordance with certain embodiments of the invention, larazotide or derivative is administered more than once daily during NSAID therapy to promote GI tight junction integrity. For example, larazotide or derivative may be administered about two times daily, or about three times daily during NSAID therapy. In some embodiments, the patient takes an NSAID daily, and takes larazotide or derivative from one to three times daily.

Other aspects and embodiments of the invention will be apparent from the following detailed description.

### BRIEF DESCRIPTION OF DRAWINGS

FIG. 1 depicts a response of ischemia-injured porcine jejunum to larazotide and indomethacin via measurement of transepithelial electrical resistance (TER) over time. The controls include a non-ischemic control (uninjured tissue) with indomethacin and an ischemic control (no Larazotide) with indomethacin. In addition, larazotide doses of 0.1 μM, 1 μM and 10 μM were administered along with indomethacin. The 1 μM dose of larazotide demonstrated increased TER in the presence of indomethacin.

FIG. 2 shows that the larazotide recovery response is dose dependent, with a reduction of effect at lower or higher doses. FIG. 2 further shows that the larazotide fragments GVLVQPG (SEQ ID NO:2) and VLVQPG (SEQ ID NO:3) can inhibit the larazotide recovery response.

### DETAILED DESCRIPTION

The present invention provides compositions and methods for treating or preventing gastrointestinal damage associated with acute or chronic non-steroidal anti-inflammatory drug (NSAID) therapy, including, for example, NSAID-induced gastropathy or enteropathy. In various embodiments, the invention relates to administering a regimen of larazotide (or a derivative) to a patient to repair or prevent injury to mucosal tissue of the gastrointestinal epithelium. In accordance with the invention, larazotide functions to repair injured mucosal tissue or prevent injury to mucosal tissue in a patient undergoing acute or chronic NSAID therapy.

In various embodiments, the patient has or is at risk of developing NSAID-induced gastropathy or enteropathy, for example, based on acute or chronic NSAID use. As used herein, the term “chronic NSAID use” or “chronic NSAID therapy” refers to administration of NSAID therapy a plurality of times per week for at least one month. In some embodiments, the patient takes NSAID therapy from two to fourteen times per week. For example, the patient may take an NSAID at least once daily (on average), or at least two times daily (on average), or at least seven times weekly (on average), or at least fourteen times weekly (on average). In various embodiments, the patient undergoes this NSAID regimen for at least one month, at least two months, at least four months, at least six months, or more. As used herein, “acute NSAID use” or “acute NSAID therapy” refers to administration of an NSAID a plurality of times, but the acute regimen has a shorter duration than chronic NSAID therapy. For example, acute NSAID therapy includes administration of an NSAID at least once daily for one or more days, including for at least one week, or at least two weeks (but less than one month). Acute therapy can be intermittent.

NSAIDs cause injury to the mucosal lining of the GI tract and systemic effects by inhibiting the cyclooxygenase (COX) enzymes resulting in prostaglandin depletion. Tissue prostaglandins are produced via two distinct enzyme-dependent pathways: a COX-1 and a COX-2 pathway. Prostaglandins derived from the COX-1 enzyme mediate gastroduodenal protection, renal perfusion, and platelet activity. The COX-2 pathway, in contrast, is inducible by inflammatory stimuli and produces mediators of inflammation, pain, and fever. NSAIDs work by blocking COX-1 and COX-2 enzymes throughout the body. Thus, inhibition of the COX-1 pathway blocks production of prostaglandins that play a protective role in the GI by increasing mucosal blood flow, stimulating the synthesis and secretion of mucus critical for the mucosal lining, and promoting proliferation of the epithelial lining. Secretion of mucus and epithelial proliferation are critical aspects to gut health in order to prevent intestinal permeability or leaky gut. A major consequence of prostaglandin depletion is to create an environment that is conducive to peptic ulcer formation, bleeding and serious GI complications. Since prostaglandins are essential to both the maintenance of intact GI defenses and normal platelet function, NSAIDs, including aspirin, promote ulcer formation as well as bleeding.

In some embodiments, the patient receives chronic NSAID therapy for chronic pain, including but not limited to arthritis, osteoarthritis, migraine, back pain, joint pain, or autoimmune disease. In some embodiments, the patient received chronic NSAID therapy for reducing thrombosis, including for prevention of heart attack and stroke. Administration of larazotide or derivative in these patients can reduce gastrointestinal damage associated with the chronic NSAID therapy. As shown herein, the mechanism of larazotide-induced recovery of intestinal mucosa is not dependent on prostaglandin production, and thus larazotide (or derivative) can be effective even in a condition of prostaglandin depletion in the gastrointestinal mucosa.

Non-limiting examples of NSAIDs include indomethacin, aspirin, ibuprofen, naproxen, and mesalamine. In some embodiments, the NSAID is aspirin, ibuprofren, or naproxen.

In some embodiments, the patient is exhibiting one or more symptoms of NSAID-induced gastropathy or enteropathy, or gastrointestinal mucosal tissue damage, including, but not limited to, lower gastrointestinal complications, such as ulcer, bleeding, perforation, obstruction, epigastric pain, nausea, indigestion, constipation, and abdominal distension. In some embodiments, the patient exhibits one or more symptoms of side effects related to acute NSAID use, including, but not limited to, gas, bloating, heartburn, stomach pain, nausea, vomiting, and diarrhea.

Larazotide is a peptide agent that promotes tight junction integrity in the gastrointestinal tract (GI). Larazotide has the amino acid sequence: Gly Gly Val Leu Val Gln Pro Gly (SEQ ID NO:1), and can be formulated for systemic or targeted release in portions of the GI (e.g., stomach, small intestine and/or large intestine). Larazotide has been shown in clinical trials to exhibit benefit at reducing intestinal disease symptoms, particularly at lower doses (e.g., 0.5 mg dose). See US 2016/0022760, which is hereby incorporated by reference in its entirety. Higher doses (e.g., 1 mg and 2 mg doses) showed an attenuation of activity, or no activity at all. It is believed that an aminopeptidase located within the brush borders of the lumen surface may create larazotide-derived fragments, including fragments missing N-terminal glycine residues. For example, the fragments GVLVQPG (SEQ ID NO:2) and VLVQPG (SEQ ID NO:3) are largely inactive as tight junction regulators. Moreover, when these two fragments are mixed with full length larazotide, activity is completely abolished. Local buildup of these inactive larazotide fragments (due to excessive larazotide) may in fact compete and block function of the peptide. This would explain clinical observations that low doses of larazotide work best by avoiding the reservoir of competing inactive fragments. Thus, in some embodiments, controlled release or sustained release formulations are employed to increase effectiveness of larazotide or derivative.

In some embodiments, the patient receives a larazotide derivative, for example, having one or more amino acid substitutions, deletions, and/or insertions with respect to SEQ ID NO:1. For example, the derivative may have 1, 2, 3, 4, or 5 amino acid deletions, insertions, and/or substitutions with respect to SEQ ID NO:1. Exemplary larazotide derivatives are described in U.S. Pat. No. 8,785,374, U.S. Pat. No. 8,957,032, and U.S. Pat. No. 9,279,807, which are hereby incorporated by reference in their entirety. In some embodiments, the derivative has one or more non-genetically encoded amino acids, or one or more D-amino acids. In some embodiments, the larazotide derivative is a retro-inverso larazotide peptide or derivative thereof. The term “larazotide” or “larazotide treatment” refers to treatment with larazotide or a derivative that promotes tight junction integrity.

Larazotide (or derivative) may be administered in any suitable form, including as a salt. For example, larazotide may be administered as an acetate salt. Salts of larazotide, including the acetate salt and hydrochloride salt, are described in US 2013/0281384, which is hereby incorporated by reference in its entirety. Alternative salts may be employed, including any pharmaceutically acceptable salt of the peptide such as those listed in Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In various embodiments, the present invention provides pharmaceutical compositions comprising the larazotide or derivative in various formulations. Pharmaceutical composition can take the form of tablets, pills, pellets, capsules, capsules containing liquids, capsules containing multiparticulates, powders, solutions, emulsions, drops, suspensions, delayed-release formulations, sustained-release formulations, controlled-release formulations, or any other form suitable for use.

In various embodiments, the larazotide is administered in a sustained release or controlled release formulation. The sustained release or controlled release formulation avoids accumulation of inactive fragments that may act as competitive inhibitors. For example, the formulation may comprise or deliver and/or functionally release from 0.5 to about 5 mg of larazotide or derivative, or from about 0.5 to about 4 mg of larazotide or derivative, or from about 0.5 to about 3 mg of larazotide or derivative, or from about 0.5 to about 2 mg of larazotide or derivative, or from about 0.5 to about 1 mg of larazotide or derivative. In various embodiments, the sustained release or controlled release formulation contains at least 1 mg or at least 2 mg of larazotide or derivative. For example, the formulation may contain from about 1 mg to about 5 mg of larazotide or derivative, or about 1 mg to about 3 mg of larazotide or derivative.

The sustained or controlled release formulation may functionally release peptide over the course of at least about 2 hours, or over the course of at least about 2.5 hours, or over the course of at least about 3 hours, or over the course of at least about 4 hours, or over the course of at least about 5 hours in simulated intestinal fluid. The term “functional release” refers to the release of larazotide or derivative such that the peptide can interact with cells of the intestinal epithelium to promote tight junction assembly. In various embodiments, larazotide is formulated as a plurality of particles that release larazotide at different times in intestinal fluid, or at different locations in the gastrointestinal tract. In other embodiments, the formulation releases larazotide in a form that provides for a local sustained release at one or more locations, including sustained release from particles, gels, emulsions, or biodegradable matrix. In some embodiments, the sustained or controlled release composition (e.g., comprising peptide-containing particles, gels, emulsions, or biodegradable matrix) begins to release peptide starting within about 5 minutes to about 30 minutes of exposure to simulated intestinal fluid, with release of peptide continuing for at least about 180 minutes, or at least about 210 minutes, or at least about 240 minutes, or at least about 280 minutes of exposure to simulated intestinal fluid. Release profiles can be prepared, for example, using particles with different enteric polymer coats and/or different thicknesses of the polymer coats. Exemplary particles are described herein.

In one embodiment, the composition comprising peptide remains essentially intact, or may be essentially insoluble, in gastric fluid. The stability of a gastric-resistant coating can be pH dependent. Delayed-release coatings that are pH dependent will be substantially stable in acidic environments (pH 5 or less), and substantially unstable in near neutral to alkaline environments (pH greater than 5). For example, a delayed-release coating can be employed that will essentially disintegrate or dissolve in near neutral to alkaline environments such as are found in the small intestine. Examples of simulated gastric fluid and simulated intestinal fluid include, but are not limited to, those disclosed in the 2005 Pharmacopeia 23NF/28USP in Test Solutions and/or other simulated gastric fluids and simulated intestinal fluids known to those of skill in the art, for example, simulated gastric fluid and/or intestinal fluid prepared without enzymes.

Alternatively, the stability of the delayed-release coating can be enzyme-dependent. Delayed-release coatings that are enzyme dependent will be substantially stable in fluid that does not contain a particular enzyme and substantially unstable in fluid containing the enzyme. The delayed-release coating will essentially disintegrate or dissolve in fluid containing the appropriate enzyme. Enzyme-dependent control can be brought about, for example, by using materials which release the active ingredient only on exposure to enzymes in the intestine, such as galactomannans.

In some embodiments, the formulation releases larazotide or derivative in simulated gastric fluid, so as to release larazotide or derivative in the stomach. For example, a population of peptide-coated particles can be employed having an outer coating that disintegrates in simulated gastric fluid.

Various formulations can be employed to deliver the larazotide or derivative to a location of interest. For example, the compositions may be formulated for targeted delivery to the gastrointestinal tract including the stomach, small intestine, large intestine and rectum including all subsections thereof. By targeting release of larazotide or derivative in the affected region(s) (e.g. stomach, duodenum, jejunum, ileum, colon transversum, colon descendens, colon ascendens, colon sigmoidenum and cecum), tight junction integrity at various portions of the GI can be improved or protected during NSAID therapy.

In some embodiments, the composition is formulated to release in the small intestine, including one or more of the duodenum, jejunum, and/or the ileum, and optionally the stomach. Alternatively, or in addition, the composition is formulated to release in the large intestine, including one or more of the cecum, the ascending colon, the transverse colon, the descending colon, and/or the sigmoid colon.

In various embodiments, the composition may be formulated to have sustained-release profiles, i.e. slow release of the larazotide in the GI tract over an extended period of time. In various embodiments, the composition may be formulated to have a delayed-release profile, i.e. not immediately release the larazotide upon ingestion; rather, postponement of the release until the composition is lower in the gastrointestinal tract; for example, for release in the small intestine (e.g., one or more of duodenum, jejunum, ileum) and/or the large intestine (e.g., one or more of cecum, ascending, transverse, descending or sigmoid portions of the colon, and rectum). In an embodiment, the pharmaceutical composition is formulated to have a delayed-release profile as described in, for example, U.S. Pat. No. 8,168,594, the entire contents of which are hereby incorporated by reference.

For example, the larazotide or derivative may be administered to the duodenum of the patient, as an oral dosage, delayed-release composition that contains larazotide (or derivative)-coated beads that are stable in gastric fluid and unstable in intestinal fluid so as to substantially release the peptide in the duodenum. The composition may further comprise a second population of beads with a pH-dependent coating to affect release of the peptide in the jejunum of the patient. For example, the second population of beads may release the larazotide or derivative about 30 minutes after the beads releasing peptide in the duodenum (based on release in simulated intestinal fluid). The composition may further comprise a third population of beads with a pH-dependent coating to affect release of the peptide in the ileum of the patient. For example, the third population of beads may release the larazotide or derivative at least about 30 minutes after the beads releasing peptide in the jejunum (based on release in simulated intestinal fluid). The oral dosage composition can be in the form of a capsule or tablet. The pH-dependent coating in some embodiments is a 1:1 co-polymer of methacrylic acid and ethyl acrylate, wherein the thickness of the layer determines the release profile of each bead. In these or other embodiments, the formulation comprises a population of beads that release larazotide or derivative in the stomach, i.e., release larazotide or derivative when exposed to simulated gastric fluid. The beads may have one or more additional coatings such as a base coat, a separating layer, and an overcoat layer.

In an exemplary oral dosage composition, an effective amount of larazotide (e.g., as the acetate salt) is provided in first delayed-release particles that are capable of releasing larazotide or derivative in the duodenum of a patient, and second delayed release particles that are capable of releasing larazotide or derivative in the jejunum of a patient, and optionally a third delayed release particle capable of releasing larazotide or derivative in the stomach and/or ileum of a patient. Each particle may have a core particle, a coat comprising larazotide or derivative over the core particle, and a delayed-release coating (e.g., a 1:1 co-polymer of acrylate and methacrylate) outside the coat comprising larazotide or derivative. Whereas the first delayed-release particles release at least 70% of the larazotide or derivative in the first delayed-release particles by about 60 minutes of exposure to simulated intestinal fluid having a pH of greater than 5; the second delayed-release particles release at least 70% of the larazotide or derivative by about 30 and about 90 minutes of exposure to simulated intestinal fluid having a pH of greater than 5. The third delayed-release particles (for release in the ileum) release at least 70% of the larazotide or derivative by about 120 minutes to about 240 minutes (e.g., about 120 minutes to about 180 minutes) of exposure to simulated intestinal fluid. For release in the stomach, peptide will release in the presence of simulated gastric fluid.

In some embodiments where the damage to the colon is involved, the larazotide or derivative may be administered to the colon of a patient, as an oral dosage, modified-release composition. Various colon-specific delivery approaches may be utilized. For example, the modified release formulation may be formulated using a colon-specific drug delivery system (CODES) as described for example, in Li et al., AAPS PharmSciTech (2002), 3(4): 1-9, the entire contents of which are incorporated herein by reference. Drug release in such a system is triggered by colonic microflora coupled with pH-sensitive polymer coatings. For example, the formulation may be designed as a core tablet with three layers of polymer. The first coating is an acid-soluble polymer (e.g., EUDRAGIT E), the outer coating is enteric, along with a hydroxypropyl methylcellulose barrier layer interposed in between. In another embodiment, colon delivery may be achieved by formulating the larazotide or derivative with specific polymers that degrade in the colon such as, for example, pectin. The pectin may be further gelled or crosslinked with a cation such as a zinc cation. Additional colon specific formulations include, but are not limited to, pressure-controlled drug delivery systems (prepared with, for example, ethylcellulose) and osmotic controlled drug delivery systems (i.e., ORDS-CT).

In various embodiments, the compositions of the present invention may use one or more modified-release coatings such as delayed-release coatings to provide for effective, delayed yet substantial delivery of the larazotide or derivative to the GI tract. For example, a composition can be enteric coated to delay release of the larazotide or derivative until it reaches the small intestine or the large intestine.

In an embodiment, the composition may remain essentially intact, or may be essentially insoluble, in gastric fluid. In some embodiments, the stability of the delayed-release coating can be pH dependent. Delayed-release coatings that are pH dependent will be substantially stable in acidic environments (pH of about 5 or less), and substantially unstable in near neutral to alkaline environments (pH greater than about 5). For example, the delayed-release coating may essentially disintegrate or dissolve in near neutral to alkaline environments such as are found in the small intestine (e.g. one or more of the duodenum, jejunum, and ileum) and/or large intestine (e.g. one or more of the cecum, ascending colon, transverse colon, descending colon, and sigmoid colon).

In some embodiments, the delayed-release coating (including for sustained release and controlled release formulations) includes an enteric agent that is substantially stable in acidic environments and substantially unstable in near neutral to alkaline environments. In an embodiment, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymer include, for example, EUDRAGIT® FS 30D, L 30 D-55, L 100-55, L 100, L 12,5, L 12,5 P, RL 30 D, RL PO, RL 100, RL 12.5, RS 30 D, RS PO, RS 100, RS 12,5, NE 30 D, NE 40 D, NM 30 D, S 100, S 12,5, and S 12,5 P. In some embodiments, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 and S 12,5 P is used. The enteric agent may be a combination of the foregoing solutions or dispersions.

In another embodiment, the delayed-release coating may degrade as a function of time when in aqueous solution without regard to the pH and/or presence of enzymes in the solution. Such a coating may comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings may be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, EUDRAGIT NE®, polyvinyl esters, polyvinyl acetals, polyacrylic acid esters, butadiene styrene copolymers, and the like.

In some embodiments, the stability of the pharmaceutical composition can be enzyme-dependent. Delayed-release coatings that are enzyme dependent will be substantially stable in fluid that does not contain a particular enzyme and substantially unstable in fluid containing the enzyme. The delayed-release coating will essentially disintegrate or dissolve in fluid containing the appropriate enzyme. Enzyme-dependent control can be brought about, for example, by using materials which release the active ingredient only on exposure to enzymes in the intestine. In certain embodiments, the stability of the composition can be dependent on the presence of a microbial enzyme present in the gut flora. Accordingly, in various embodiments, the delayed-release coating is degraded by a microbial enzyme present in the gut flora. In an embodiment, the delayed-release coating is degraded by a bacteria present in the small intestine. In another embodiment, the delayed-release coating is degraded by a bacteria present in the large intestine.

The present invention also provides for compositions that release multiple doses of the larazotide or derivative along the gastrointestinal tract. For example, the composition and/or formulation can release multiple doses of the larazotide or derivative at different locations along the intestines, at different times, and/or at different pH. The overall release profile of such a formulation may be adjusted using, for example, multiple particle types or multiple layers. For example, in one embodiment, a first dose of the larazotide or derivative may be formulated for release in, for example, the small intestine (e.g., one or more of duodenum, jejunum, ileum), whereas a second dose is formulated for delayed release in, for example, the large intestines (e.g., one or more of cecum, ascending, transverse, descending or sigmoid portions of the colon, and rectum). In various embodiments, the composition and/or formulation may release at least three doses, at least four doses, or at least five doses of the larazotide or derivative at different locations along the intestines, at different times, and/or at different pH.

Larazotide or derivative can be administered in unit dosage forms (e.g., tablets, capsules, or solutions). For example, larazotide or derivative (or salt thereof) can be administered at from about 0.1 mg to about 5 mg, or at from about 0.1 mg to about 2 mg, or at from about 0.25 mg to about 1 mg, or at from about 0.5 mg to about 1 mg, or at from about 0.25 to about 0.75 mg. In various embodiments, the unit dose contains at least 1 mg of larazotide or derivative, or contains at least 1.5 mg or at least 2 mg of larazotide or derivative.

In accordance with certain embodiments of the invention, larazotide or derivative is administered more than once daily during NSAID therapy to promote GI tight junction integrity. For example, larazotide or derivative may be administered about two times daily, about three times daily, about four times daily, or about five times daily during NSAID therapy. In some embodiments, the patient takes an NSAID daily, and takes larazotide or derivative from one to three times daily.

In some embodiments, including for patients exhibiting symptoms of gastrointestinal damage (e.g., NSAID-induced enteropathy) the patient further receives a probiotic. Probiotics suitable for use in the present invention include, but are not limited to, Saccharomyces boulardii; Lactobacillus rhamnosus GG; Lactobacillus plantarum 299v; Clostridium butyricum M588; Clostridium difficile VP20621 (non-toxigenic C. difficile strain); combination of Lactobacillus casei, Lactobacillus acidophilus (Bio-K +CL1285); combination of Lactobacillus casei, Lactobacillus bulgaricus, Streptococcus thermophilus (Actimel); combination of Lactobacillus acidophilus, Bifidobacterium bifidum (Florajen3); combination of Lactobacillus acidophilus, Lactobacillus bulgaricus delbrueckii subsp. bulgaricus, Lactobacillus bulgaricus casei, Lactobacillus bulgaricus plantarum, Bifidobacterium longum, Bifidobacterium infantis, Bifidobacterium breve, and Streptococcus salivarius subsp.thermophilus (VSL#3)).

Other aspects and embodiments will be apparent from this detailed description and examples below.

### EXAMPLES

### Example: Larazotide Enhances Recovery of Injured Intestinal Tissue in the Presence of NSAIDs.

In order to determine whether Larazotide could function as a therapeutic agent that could stimulate intestinal tissue repair in the presence of a non-steroidal anti-inflammatory drug (NSAID), recovery of barrier function in ischemic-injured porcine jejunum was monitored in the presence of the NSAID indomethacin. Transepithelial electrical resistance is often used to monitor barrier function due to its high sensitivity as an indicator.

Specifically, Yorkshire-cross pigs of 6-8 weeks of age were anesthetized, followed by midline laparotomy and creation of a series of 10 cm intestinal loops commencing proximal to the ileum by ligating the intestinal lumen.

The local mesenteric vasculature was ligated to select treatment loops for a period of 45 minutes. Other loops were left as non-ischemic controls. The loops were subsequently resected, and the mucosal tissues were stripped in oxygenated (95% O₂/5 % CO₂) rings from the muscle layers in preparation of ex vivo incubation in Ussing chambers.

Select tissues (n=8) were pre-treated with indomethacin (5 μM) on both sides of the tissue, prior to receiving 0.1-10 μM Larazotide on the luminal surface after 30 minutes of incubation in the Ussing chambers. Indomethacin was chosen due to the general consideration that it is a “gold standard” due to its complete inhibition of COX enzymes.

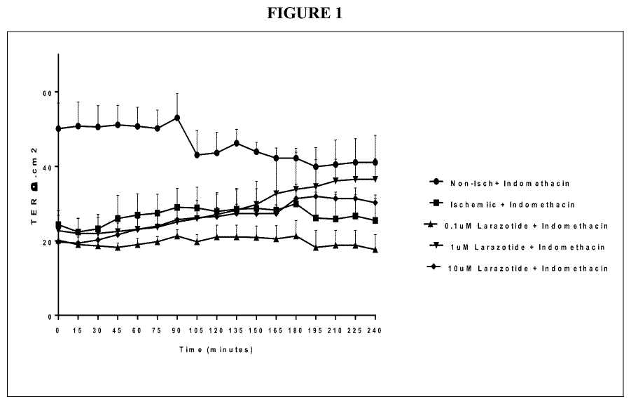

Transepithelial electrical resistance (TER) was monitored in the tissues for 240 minutes. At the end of the 240 minute recovery period, tissues were taken for histology and immunofluorescence evaluation of tight junction proteins. As shown in FIG. 1, Larazotide-induced recovery of tissue repair (by way of increased TER) was not inhibited by indomethacin. However, the tissue exhibits a decline in TER when indomethacin is applied without Larazotide.

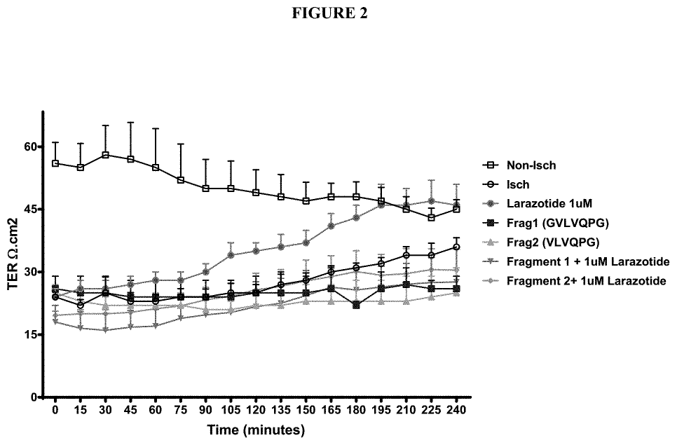

The Larazotide recovery response is dose dependent, with a reduction of effect at lower or higher doses. The higher doses may generate competitive peptide fragments. As shown in FIG. 2, larazotide fragments can inhibit the larazotide recovery response.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

1. A method for treating or preventing gastrointestinal damage associated with non-steroidal anti-inflammatory drug (NSAID) therapy in a patient, comprising administering larazotide or derivative thereof to said patient.

2. The method of claim 1, wherein the NSAID therapy is acute NSAID therapy.

3. The method of claim 2, wherein the patient takes an NSAID at least once daily for a plurality of days.

4. The method of claim 1, wherein the NSAID therapy is chronic NSAID therapy.

5. The method of claim 4, wherein the patient takes NSAID therapy at least once daily.

6. The method of any one of claims 4 to 5, wherein the patient receives chronic NSAID therapy for chronic pain.

7. The method of claim 6, wherein the chronic pain is associated with arthritis, osteoarthritis, migraine, back pain, joint pain, or autoimmune disease.

8. The method of claim 4 or 5, wherein the patient receives chronic NSAID therapy to prevent or reduce thrombosis.

9. The method of any one of claims 1 to 8, wherein the patient has NSAID-induced gastropathy or enteropathy.

10. The method of any one of claims 1 to 9, wherein the NSAID is indomethacin, aspirin, ibuprofen, naproxen, and mesalamine.

11. The method of claim 10, wherein the NSAID is aspirin, ibuprofen, and naproxen.

12. The method of any one of claims 1 to 11, comprising administering larazotide.

13. The method of any one of claims 1 to 11, comprising administering a larazotide derivative.

14. The method of any one of claims 1 to 13, wherein the larazotide is administered in a sustained release or controlled release formulation.

15. The method of claim 14, wherein the sustained release or controlled release formulation contains at least 1 mg of larazotide or derivative.

16. The method of claim 14 or 15, wherein the sustained or controlled release formulation releases peptide over the course of at least about 2 hours.

17. The method of any one of claims 14 to 16, wherein the formulation releases larazotide in a form that provides for a local sustained release at one or more locations, including sustained release from particles, gels, emulsions, or biodegradable matrix.

18. The method of claim 16 or 17, wherein the sustained or controlled release formulation begins to release peptide starting within about 5 minutes to about 30 minutes of exposure to simulated intestinal fluid, with release of peptide continuing for at least about 180 minutes.

19. The method of any one of claims 1 to 17, wherein the formulation releases larazotide or derivative in the stomach.

20. The method of any one of claims 1 to 19, wherein the larazotide or derivative is administered more than once daily during NSAID therapy.


SEQUENCE LISTING (3 sequences)

SEQ ID NO: 1 - protein, 8 aa, Artificial Sequence
  Synthetic polypeptide
GGVLVQPG

SEQ ID NO: 2 - protein, 7 aa, Artificial Sequence
  Synthetic polypeptide
GVLVQPG

SEQ ID NO: 3 - protein, 6 aa, Artificial Sequence
  Synthetic polypeptide
VLVQPG